(19) United States
(12) Patent Application Publication
(10) Pub. No.: US 2025/0195759 A1
(43) Pub. Date: Jun. 19, 2025

(54) HIGHLY INTEGRATED DRUG INFUSION DEVICE

(71) Applicant: MEDTRUM TECHNOLOGIES INC., Shanghai (CN)

(72) Inventor: Cuijun Yang, Shanghai (CN)

(73) Assignee: MEDTRUM TECHNOLOGIES INC., Shanghai (CN)

(21) Appl. No.: 18/846,276

(22) PCT Filed: Aug. 3, 2022
(86) PCT No.: PCT/CN2022/110056

§ 371 Date: Sep. 12, 2024

(30) Foreign Application Priority Data
Mar. 15, 2022 (WO) PCT/CN2022/080845

(51) Int. Cl. A61M 5/158; A61M 5/172

(52) U.S. Cl. CPC A61M 5/158; A61M 5/1723; A61M 2205/0216; A61M 2205/0233

(57) ABSTRACT

A highly integrated drug infusion device includes an infusion unit to deliver drugs; a program unit including an input end and an output end, and the input end includes a plurality of electrically connection areas for receiving the signal of analyte data, after the output end is electrically connected to the infusion unit, the program unit controls the drug delivering; an infusion cannula provided with electrodes and electrode contact point; and a conductor, one end is fixed and electrically connected to the electrode contact point, and the other end is slidably and electrically connected to the electrical connection area, when the infusion cannula is installed in the working position, the infusion cannula is connected with the infusion unit, the drug can be injected into the body through the infusion cannula, and the conductor is electrically connected to the electrical connection area, inputting signal of analyte data to the program unit.

### CROSS-REFERENCE TO RELATED APPLICATION

This application claims the priority benefit of PCT application no. PCT/CN2022/080845, filed on Mar. 15, 2022. The entirety of the above-mentioned patent application is hereby incorporated by reference herein and made a part of this specification.

### TECHNICAL FIELD

The present invention mainly relates to the field of medical instruments, in particular to a highly integrated drug infusion device.

### BACKGROUND

Diabetes is mainly a metabolic disease caused by abnormal human pancreatic function. Diabetes is a lifelong disease. At present, medical technology cannot cure diabetes. It can only control the occurrence and development of diabetes and its complications by stabilizing blood glucose. The normal human pancreas automatically monitors changes in the body's blood glucose levels and automatically secretes the required insulin. At present, the medical device for stabilizing blood glucose works by dynamically monitoring the blood glucose changes of the human body by a glucose sensor implanted in the subcutaneous tissue of the human body; and continuously accurately infusing insulin into the subcutaneous tissue of the human body through a medical cannula implanted in the subcutaneous tissue of the human body.

At present, there are some devices that can integrate the sensor probe and the infusion cannula into one device, so the detection and infusion can be completed at the same time by puncturing at one position. However, because the electrode contacting point (PAD) set on the surface of the infusion cannula is slidingly connected with the electrical connection area of the program unit to receive the body fluid analyte parameter signal, and the PAD is very thin, will be damaged easily during the sliding process, thus affecting the electrical connection stability and service life of the integrated drug infusion device.

Therefore, there is a need in the prior art for a highly integrated drug infusion device that can increase the stability of the electrical connection and prolong the service life.

### BRIEF SUMMARY OF THE INVENTION

Embodiments of the present invention disclose a highly integrated drug infusion device, the infusion device is provided with a conductor, one end of the conductor is fixed and electrically connected to the PAD, and the other end is slidably connected to the electrical connection area of the program unit. The PAD is no longer slidably connected to the electrical connection area and will not be damaged, thereby improving the stability of the electrical connection of the drug infusion device and prolonging the service life.

The invention discloses a highly integrated drug infusion device, comprising: an infusion unit configured to deliver drugs; a program unit comprises an input end and an output end, and the input end comprises a plurality of electrically connection areas for receiving the signal of analyte data in the body fluid, after the output end is electrically connected to the infusion unit, and the program unit controls the drug delivering of the infusion unit; an infusion cannula provided with electrodes and electrode contact point; and a conductor, one end is fixed and electrically connected to the electrode contact point, and the other end is slidably and electrically connected to the electrical connection area, when the infusion cannula is installed in the working position, the infusion cannula is connected with the infusion unit, the drug can be injected into the body through the infusion cannula, and the conductor is electrically connected to the electrical connection area, inputting signal of analyte data in the body fluid to the program unit.

According to one aspect of this invention, the conductor is an elastic conductor.

According to one aspect of this invention, the elastic conductor is elastic pins.

According to one aspect of this invention, the elastic conductor is an elastic conductive member that comprises spaced conductive area and insulating area.

According to one aspect of this invention, the elastic conductive member is a hollow elastic conductive column, which is in interference fit with the infusion cannula.

According to one aspect of this invention, the elastic conductive member is rectangular solid.

According to one aspect of this invention, the elastic conductive member at least partially surrounds the infusion cannula.

According to one aspect of this invention, the conductive area and the insulating area pass through the elastic conductive member in the transverse direction, respectively.

According to one aspect of this invention, the conductive area and the insulating area surround the surface of the elastic conductive member.

According to one aspect of this invention, the conductive areas are elastic conductive members distributed at intervals, and the insulating area is the spaced air area of the conductive area.

According to one aspect of this invention, the electrical connection area is a metal conductive contact.

According to one aspect of this invention, the elastic conductor is one of conductive adhesive strips, conductive foam or conductive bubble.

According to one aspect of this invention, the highly integrated drug infusion device consists of multiple parts, the infusion unit and the program unit are arranged in different parts, and the different parts are electrically connected through a plurality of electrical contact.

According to one aspect of this invention, the conductor comprises an elastic pin and an elastic conductor including a spaced conductive area and an insulating area.

Compared with the prior arts, the technical solution of the present invention has the following advantages:

In the integrated drug infusion device disclosed herein, the infusion device is provided with a conductor, one end of the conductor is fixed and electrically connected to the PAD. The other end is slidably connected to the electrical connection area of the program unit. The sliding connection between the conductor and the electrical connection area replaces the sliding connection between the PAD and the electrical connection area. Thus the PAD will not be damaged. The conductor's thickness is much larger than that of PAD, and its wear resistance is also much larger than that of PAD, so it increases the electrical connection stability and prolongs the service life of the highly integrated drug infusion device.

Furthermore, the conductor is an elastic conductor, and the elastic material deforms to lock after being squeezed. The elastic conductor can enhance the stability of the contact and act as a buffer. Therefore, the elastic conductors can be connected to each other more closely as a conductive member or as an auxiliary member of an electrical connection position, thereby improving the reliability of the electrical connection.

Furthermore, the elastic conductor is an elastic conductive member that comprises conductive areas and insulating areas distributed at intervals, which can realize the conduction between circuits and the insulation between different circuits at the same time, making the deployment of the infusion device more compact and convenient for the integrated design of the infusion device.

Furthermore, the conductor comprises elastic pins and/or an elastic conductor, including a spaced conductive area and an insulating area, which can be flexibly selected according to actual needs.

Furthermore, the elastic conductive member is a hollow elastic conductive column, the interference fit between the elastic conductive column and the infusion cannula can prevent poor contact, and the electrical connection stability of the PAD and the elastic conductor 170 can be maintained even if no external pressure.

Furthermore, the infusion unit comprises a plurality of infusion subunits, the plurality of infusion subunits being electrically connected to the output end, respectively, and the program unit controlling whether each infusion subunit delivers drugs. Different drugs are reserved in different infusion subunits, and the program unit sends different drug infusion instructions to different infusion subunits to achieve precise control of the analyte level in body fluid.

### BRIEF DESCRIPTION OF THE DRAWINGS

FIG. 1 is a flow chart of the operation of a highly integrated drug infusion device according to an embodiment of the present invention.

FIG. 2a is a schematic cross-sectional view of an infusion cannula of a highly integrated drug infusion device in a pre-installation position according to one embodiment of the present invention.

FIG. 2b is a schematic cross-sectional view showing the infusion cannula of the integrated drug infusion device in a working position according to an embodiment of the present invention.

FIG. 3a is the section view of an elastic conductor, PAD and electrical connection area of FIG. 2b according to the embodiment of the invention.

FIG. 3b is the side view of the elastic conductor in FIG. 3a according to the embodiment of the invention.

FIG. 3c is another section view of an elastic conductor PAD and electrical connection area according to another embodiment of the invention.

FIG. 3d and FIG. 3e are different section view of elastic conductors, PADs and electrical connection areas in different embodiments of the invention.

FIG. 4 is the schematic diagram of an elastic conductor, a PAD and an electrical connection area according to another embodiment of the invention.

FIG. 5a-FIG. 5b are schematic diagrams of the electrical connection positions of the electrical connection area and the elastic conductor in different embodiments of the invention.

FIG. 6a-FIG. 6b are schematic diagrams of the infusion cannula, PAD and the elastic conductor in different embodiments of the invention.

FIG. 7a is a top view of a highly integrated drug infusion device 100 in accordance with another embodiment of the present invention.

FIG. 7b is a top view of a highly integrated drug infusion device 100 in accordance with another embodiment of the present invention.

FIG. 8 is a schematic view of a highly integrated drug infusion device and a remote device according to still another embodiment of the present invention.

### DETAILED DESCRIPTION

As described above, in the prior art device, PAD set on the surface of the infusion cannula is slidingly connected with the electrical connection area of the program unit to receive the body fluid analyte parameter signal, and PAD will be damaged easily during the sliding process, thus affecting the electrical connection stability and service life of the integrated drug infusion device.

In order to solve this problem, the present invention provides a highly integrated drug infusion device, the infusion device is provided with a conductor, one end of the conductor is fixed and electrically connected to the PAD, and the other end is slidably connected to the electrical connection area of the program unit. The PAD is no longer slidably connected to the electrical connection area and will not be damaged, thereby improving the stability of the electrical connection of the drug infusion device and prolonging the service life.

Various exemplary embodiments of the present invention will now be described in detail with reference to the drawings. The relative arrangement of the components and the steps, numerical expressions and numerical values set forth in the embodiments are not to be construed as limiting the scope of the invention.

In addition, it should be understood that, for ease of description, the dimensions of the various components shown in the figures are not necessarily drawn in the actual scale relationship, for example, the thickness, width, length or distance of certain units may be exaggerated relative to other structures.

The following description of the exemplary embodiments is merely illustrative, and is not intended to be in any way limiting the invention and its application or use. The techniques, methods and devices that are known to those of ordinary skill in the art may not be discussed in detail, but such techniques, methods and devices should be considered as part of the specification.

It should be noted that similar reference numerals and letters indicate similar items in the following figures. Therefore, once an item is defined or illustrated in a drawing, it will not be discussed further in the following description of the drawings.

FIG. 1 is a flow chart showing the operation of a highly integrated drug infusion device according to an embodiment of the present invention.

The highly integrated drug infusion device of the embodiment of the invention comprises three basic parts: electrodes, a program unit and an infusion unit. The body fluid analyte data is obtained by the electrodes and converted into an electrical signal. Electrical signals are passed to the program unit via electrodes and/or electrode leads. After analyzing the body fluid analyte data signal, the program unit sends a signal to the infusion unit controlling whether to perform a drug infusion, thereby stabilizing the body fluid parameters. The body fluid analyte data are detected by the electrodes in real time, and the cycle of detection and infusion is without interruption. This process does not require human intervention and is done directly through program analysis to control the stability of body fluid parameters.

In another embodiment of the present invention, after receiving the analyte parameter information, the program unit only uses the analyte parameter information as a comparison parameter index for the program unit to control whether the alarm unit issues an alarm, for example, when the detected analyte parameter information exceeds the preset threshold, the program unit controls the alarm unit to send an alarm. The program unit sends a signal to the infusion unit whether to perform drug infusion according to the preset program, to stabilize body fluid parameters.

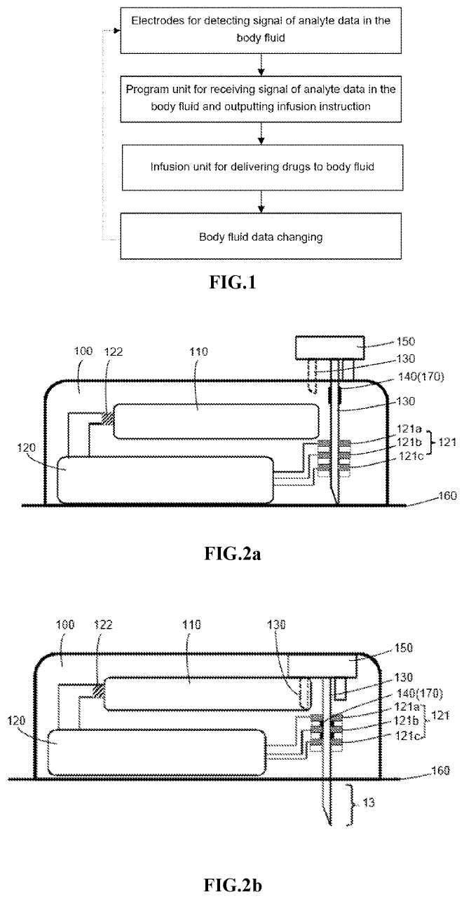

FIG. 2a-2b are cross-sectional views of a highly integrated drug infusion device 100 according to an embodiment of the present invention, and the highly integrated drug infusion device 100 is an integral structure. FIG. 2a shows the infusion cannula 130 in the pre-installation position while FIG. 2b shows the infusion cannula 130 in the working position.

Program unit 120 includes an input end 121 and an output end 122. The input end 121 is used for receiving a body fluid analyte data signal. In the embodiment of the invention, the input end 121 includes electrically connective regions 121a1,121b and 121c. When in operation, the input end 121 is electrically connected to the electrode contact point (PAD) 140 through the electrical conductor 170 to receive the parameter signal, wherein the electrical conductor 170 is fixed connected to the PAD 140, so only one part is shown in FIG. 2a and FIG. 2b. The specific connection method of the input end 121 and the PAD 140 will be described in detail below. In other embodiments of the invention, the input end 121 may only include two also electrically connective regions or include more electrically connective regions depending on the number of electrodes. The output end 122 is electrically coupled to the infusion unit 110, allowing the program unit 120 to effectively control the infusion unit 110.

In an embodiment of the invention, the infusion cannula 130 is mounted on the mounting unit 150. When the infusion cannula 130 is in the pre-installation position, the mounting unit 150 protrudes from the outer surface of the integrated drug infusion device 100, as shown in FIG. 2a. When the infusion cannula 130 is installed in the working position, the mounting unit 150 is pressed into the integrated drug infusion device 100 with the top portion integral with the integrated drug infusion device 100 housing, as shown in FIG. 2b. Prior to use by users, the mounting unit 150 holds the infusion cannula 130 in the pre-installation position. After the integrated drug infusion device 100 is attached on the surface of the human body, the mounting unit 150 is pressed to insert the infusion cannula under skin, and the integrated drug infusion device can start operation. Compared with other infusion cannula installation methods, the installation method of the embodiment of the invention reduces the steps required for installation, makes the installation more convenient and flexible and improves the user experience.

The manner of setting the infusion cannula 130 in the mounting unit 150 can be various, and is not specifically limited herein. Specifically, in the embodiment of the present invention, the other side of the mounting unit 150 also protrudes from the partial infusion cannula 130 (shown by a dotted line in FIG. 2a and FIG. 2b) for subsequent connection with the outlet of the infusion unit 110 to achieve drug circulation.

As shown in FIG. 2a, electrical conductor 170 is not electrically coupled to the input end 121 when the infusion cannula 130 is in the pre-installation position. And the other end of the infusion cannula 130 is also not connected with the infusion unit 110 outlet. As shown in FIG. 2b, when the infusion cannula 130 is mounted in the working position, one end of the infusion cannula 130 is inserted subcutaneously (indicated by the solid line portion of the infusion cannula in FIG. 2b) and the other end (illustrated by the dotted portion of the infusion cannula in FIG. 2b) is connected with the outlet of the infusion unit 110, thereby establishing a flow path for the drug from the infusion unit 110 to the body tissue fluid. At the same time, the electrical contact region 140 and the electrical conductor 170 reach the electrically connective region of the input end 121, enabling electrical connection between the program unit 120 and the electrical contact region 140.

It should be noted that even if the infusion cannula 130 and the infusion unit 110 are connected, and the input end 121 and the electrical conductor 170 of the infusion cannula 130 are electrically connected, as long as the infusion cannula 130 does not penetrate the skin, the program unit 120 will not enter working mode, so that the integrated drug infusion device does not generate any analyte data signal, nor does it issue an instruction to inject drug or send alarm. Therefore, in other embodiments of the present invention, when the infusion cannula 130 is in the pre-installation position, the electrical conductor 170 may also be electrically connected to the electrically connective region of the input end 121 or the infusion cannula 130 may be coupled to the outlet of the infusion unit 110. And there are no specific restrictions herein.

In an embodiment of the invention, a medical tape 160 for attaching the integrated drug infusion device 100 to the skin surface is used to paste the program unit 120, the infusion unit 110, the electrode and the infusion cannula 130 as a whole on the skin. When the infusion cannula 130 is installed in the working position, the portion of the infusion cannula 130 that is inserted into the skin is 13.

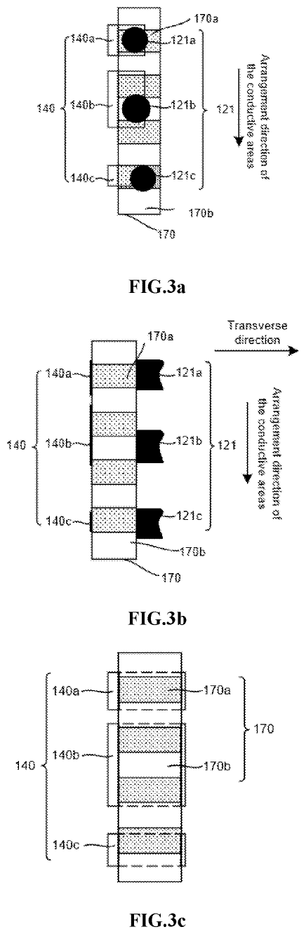

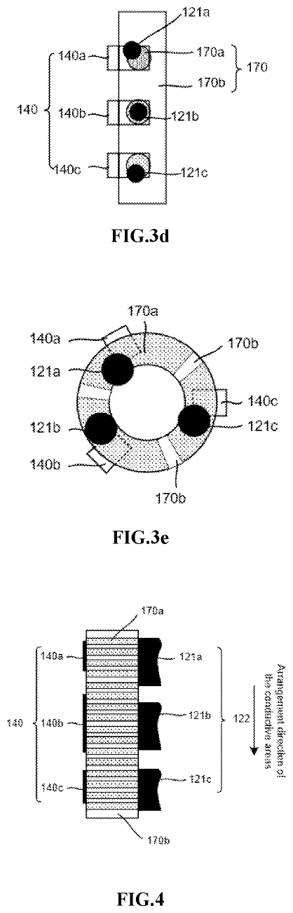

FIG. 3a is the section view of an elastic conductor, PAD and electrical connection area of FIG. 2b according to the embodiment of the invention. FIG. 3b is the side view of the elastic conductor in FIG. 3a. FIG. 3c is another section view of an elastic conductor PAD and electrical connection area according to another embodiment of the invention. FIG. 3d and FIG. 3e are different section view of elastic conductors, PADs and electrical connection areas in different embodiments of the invention.

During the use of the existing integrated drug infusion device, the infusion cannula 130 and the input end 121 will slide relative to each other. Since no additional electrical conductor is provided on the infusion cannula, the electrode contacting point (PAD) 140 will directly rub against the electrical connection area of the input end 121, while the PAD 140 is very thin and is easily damaged in the process of rubbing with the input end 121, affecting the stability of the electrical connection between the PAD 140 and the input end 121, thereby affecting the service life of highly integrated drug infusion devices.

In the embodiment of the present invention, an electrical conductor 170 is further provided on the infusion cannula 130 to realize electrical connection between the PAD 140 on the infusion cannula and the electrical connection areas 121a, 121b and 121c. When the infusion cannula 130 and the input end 121 slide relative to each other, since the conductor 170 is fixed connected to the PAD 140 on the infusion cannula 130, the component that actually rubs against the input end 121 is the electrical conductor 170, and the thickness of the electrical conductor 170 can be selected. It can be much larger than the PAD, and its degree of friction resistance is much larger than that of the PAD 140, so the stability of the electrical connection between the PAD 140 and the input end 121 can be increased.

Preferably, in the embodiment of the present invention, the conductor 170 is an elastic conductor, and the elastic material deforms to lock after being squeezed. The use of the elastic conductor can be better electrical contacted, and at the same time it can be used as a buffer. Therefore, the elastic conductors can be connected to each other more closely as a conductive member or as an auxiliary member of an electrical connection position, thereby improving the reliability of the electrical connection.

In an embodiment of the present invention, the elastic conductor is an elastic pin, the position and number of which correspond to the position and number of the PAD.

In another embodiment of the present invention, regardless of the number of PADs, the number of elastic conductors is set to one, and the provision of only one elastic conductor reduces the number of internal components of the highly integrated drug infusion device.

At this time, the elastic conductor 170 is an elastic conductive member that comprises at least two conductive area 170a and at least one insulating area 170b. The conductive area 170a and the insulating area 170b function as conductive conduction and electrical insulation, respectively. The conductive area 170a and the insulating area 170b cannot be separated from each other, that is, the conductive area 170a and the insulating area 170b belong to an integral part of the elastic conductor 170, respectively.

An insulating area 170b is arranged between adjacent conductive area 170a. Different PAD 140 or different electrical connection areas are electrically connected with different conductive area 170a respectively, so that any two PAD 140 or any two electrical connection areas are electrically insulated from each other.

Inside the elastic conductor 170, the conductive area 170a and the insulating area 170b pass through the elastic conductor 170 in the transverse direction, as shown in FIG. 3b. Here, the transverse direction refers to the direction from the PAD 140 to the corresponding electrical connection area, or the direction of the current between the PAD 140 and the electrical connection area. When the PAD 140 is electrically connected with the electrical connection area, transverse conduction of the elastic conductor 170 can be ensured, not longitudinal conduction. While the elastic conductor 170 electrically connects the PAD 140 with the corresponding electrical connection are, it also electrically insulates different PADs 140 or different electrical connection area. In other embodiments of the present invention, the conductive area and the insulating area of the elastic conductor 170 may also surround the surrounding surface of the electrical insulating material of the elastic conductor, or the conductive area may be an elastic conductor distributed at intervals, and the insulating area may be the middle space of the conductive area, such as air or vacuum. An elastic conductor 170 plays the role of conduction and electrical insulation at the same time. The complexity of the internal design of the infusion device is reduced, the internal deployment is more compact, and the electrical connection reliability of the infusion device is improved.

In the embodiment of the invention, the elastic conductor 170 can be one of the materials of conductive adhesive strip, conductive foam or conductive bubble, because these materials such as rubber strip, foam, bubble and so on is electrically insulating, which can ensure good insulation effect between different circuits, and set up a conductive area on the surface of rubber strip, foam and bubble. Alternatively, the conductive area passes through the insulating material in the transverse direction of the interval, and the circuit conduction can be realized on all surfaces of the elastic conductor 170.

It should be noted that in other embodiments of the invention, the conductive area 170a or the insulating area 170b can also have a certain inclination, or be arranged inside the elastic conductor 170 in other directions or ways. There is no specific limitation here, as long as the above conditions of conductive conduction and electrical insulation can be met.

In another embodiment of the invention, the conductive area 170a and the insulating area 170b are arranged at intervals and pass through the elastic conductor 170 respectively. In another embodiment of the invention, different conductive area 170a are arranged in the same insulating area 170b, that is, surrounded by the same insulating area 170b, as shown in FIG. 3d. In another embodiment of the invention, the top view of the elastic conductor 170 may be circular, as shown in FIG. 3e. In another embodiment of the invention, the top view of the elastic conductor 170 may also be circular.

In other embodiments of the invention, the elastic conductor 170 can also have other shapes, which are not specifically limited here, as long as the conditions for realizing the above functions of the elastic conductor 170 can be met.

Please continue to refer to FIG. 3a and FIG. 3b. When the elastic conductor 170 is electrically connected with the PAD 140 and the electrical connection area respectively, there is an insulating area 170b between any two PAD 140 connected with the elastic conductor 170. Specifically, in the embodiment of the invention, the insulating area 170b separated between any two PAD 140 comprises a part of an insulating area 170b (between 140a and 140b of FIG. 3a and FIG. 3b), or an insulating area 170b, or more than one insulating area 170b (between 140c and 140b of FIG. 3a and FIG. 3b). Similarly, the insulating area 170b separated between any two electrical connection area connected to the elastic conductor 170 comprises a part of one insulating area 170b, one insulating area 170b, or more than one insulating area 170b. However, it is obvious that the PAD and the corresponding electrical connection area (such as between 140a and 121a, between 140b and 121b, or between 140c and 121c) share a common part of the conductive area 170a to realize the conductivity of the two. The conductive area of the common part comprises a part of a conductive area 170a (as between 140c and 121c in FIG. 3a and FIG. 3b), or a conductive area 170a, or more than one conductive area 170a.

In combination with FIG. 3a and FIG. 3b, it is easy for those skilled in the art to understand that a part of the above insulating area or conductive area, an insulating area or conductive area, and more than one insulating area or conductive area are only the span range of the PAD or electrical connection area in one-dimensional direction (such as the arrangement direction of the conductive area).

In other embodiments of the invention, a part of an insulating area or conductive area, an insulating area or conductive area, and more than one insulating area or conductive area can also represent the coverage of the PAD or electrical connection area to the insulating area or conductive area in the two-dimensional direction (in area), as shown in FIG. 3c. Taking the PAD as an example, the dotted line in FIG. 3c represents the partial outline of the pin. Obviously, the PAD 140 can cover a part of an insulating area or conductive area, or an insulating area or conductive area, or more than one insulating area or conductive area.

Obviously, when the number of conductive areas or insulating areas between the above components is large or the range is wide, the reliability of electrical connection or electrical insulation between structures will be significantly improved.

Obviously, when the number of electrodes is 2, the number of PADs and electrical connection areas also will be 2. At this time, the elastic conductor 170 only comprises two conductive area 170a and an insulating area 170b arranged between the two conductive area 170a. That is, two pairs of different PADs and electrical connection areas are electrically connected through different conductive area 170a to realize conductivity. At the same time, two PADs or two electrical connection areas are separated by insulation areas to achieve electrical insulation.

FIG. 4 is the schematic diagram of an elastic conductor, a PAD and an electrical connection area according to another embodiment of the invention.

The other embodiments of the invention may also comprise more electrodes. Therefore, the elastic conductor 170 comprises more conductive areas and insulating areas arranged at intervals, and the mode of electrical connection will be more flexible, as shown in FIG. 4.

It should be noted that in other embodiments of the invention, there are at least three electrodes, that is, at least three PADs, of which at least two PADs are electrically connected with the corresponding electrical connection area through different conductive area 170a, and the connection method and principle are consistent with the above. For other PADs and electrical connection areas not connected with the elastic conductor 170, the embodiment of the invention does not limit the connection mode or connection principle. For example, in one embodiment of the invention, only the working electrode and the counter electrode are electrically connected with the electrical connection area through the above elastic conductor through the corresponding PADs, while the reference electrode is electrically connected with the electrical connection area through other ways, such as through the aforementioned elastic pins to connect with the electrical connection area.

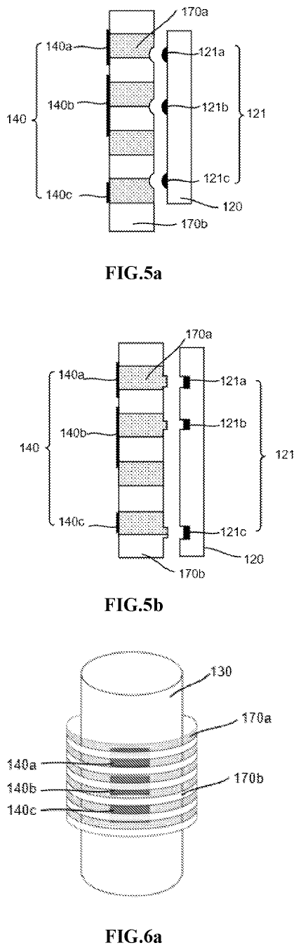

FIG. 5a-FIG. 5b are schematic diagrams of the electrical connection positions of the electrical connection area and the elastic conductor in different embodiments of the invention.

For convenience of annotation and description, the electrical connection area 121 and the elastic conductor 170 in FIG. 5a and FIG. 5b will be shown separately.

As shown in FIG. 5a, in the embodiment of the invention, the electrical connection area 121a, 121b, 121c is a convex spherical crown metal conductive contact. Correspondingly, the elastic conductor 170 is provided with a concave part at the position connected with the convex metal conductive contact to make the connection closer. At the same time, the connection between the convex part and the concave part also plays a role in fixing the position of the elastic conductor 170, that is, no matter what external force the infusion device receives, the position of the elastic conductor 170 is always fixed without displacement, so as to ensure that the elastic conductor 170 performs normal conduction and insulation work.

It should be noted that the elastic conductor 170 may not design a concave part. When pressed by the protruding metal conductive contact, the concave part matching with the metal conductive contact will automatically appear on the elastic conductor 170 to ensure the function of electrical connection or electrical insulation.

As shown in FIG. 5b, in another embodiment of the invention, the electrical connection area is arranged inside the input end 121. At this time, the elastic conductor 170 is correspondingly provided with a convex part, which can enter the interior of the input end 121 and be electrically connected with the corresponding electrical connection area.

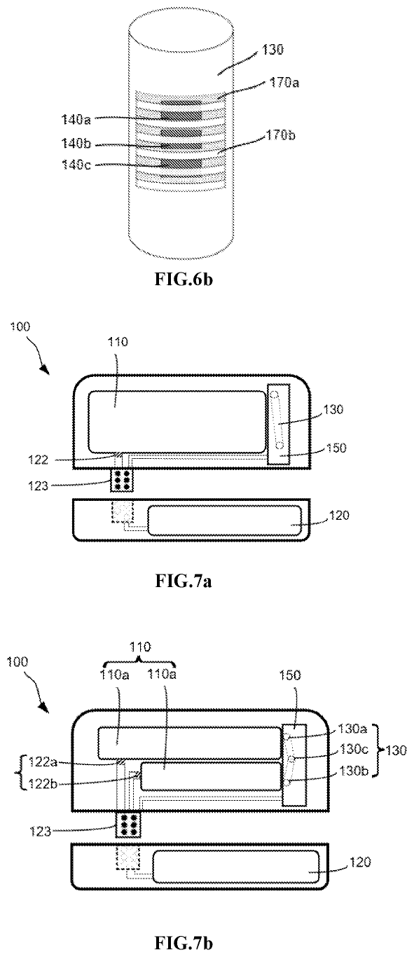

FIG. 6a-FIG. 6b are schematic diagrams of the infusion cannula, PAD and the elastic conductor in different embodiments of the invention.

Since the infusion device is a highly integrated infusion device, the electrodes are formed on the circular infusion cannula 130, and the PAD 140 is also formed on the infusion cannula 130 with a certain arc. Therefore, in an embodiment of the present invention, the elastic conductive member is a hollow elastic conductive column, the interference fit between the elastic conductive column and the infusion cannula can prevent poor contact, and the electrical connection stability of the PAD 140 and the elastic conductor 170 can be maintained even if no external pressure, as shown in the FIG. 6a. In another embodiment of the present invention, the elastic conductor 170 is a rectangular solid, which surrounds the infusion cannula 130, that is, a deployment similar to that the infusion cannula 130 inserted into the elastic conductor 170 is formed, so that the PAD 140 and the elastic conductor 170 are completely fitted. Under the pressure of the input end 121 or other components such as the housing, the PAD 140, the elastic conductor 170 and the electrical connection area are locked to achieve stable electrical connection. In yet another embodiment of the present invention, the elastic conductor 170 only partially surrounds the infusion cannula 130, and the contact length between the elastic conductor 170 and the infusion cannula 130 is not less than the length of the PAD 140, that is, the elastic conductor 170 completely covers the PAD 140, as shown in FIG. 6b, under the pressure of the input end 121 or other components such as the housing, the PAD 140, the elastic conductor 170 and the electrical connection area can also be locked to achieve stable electrical connection

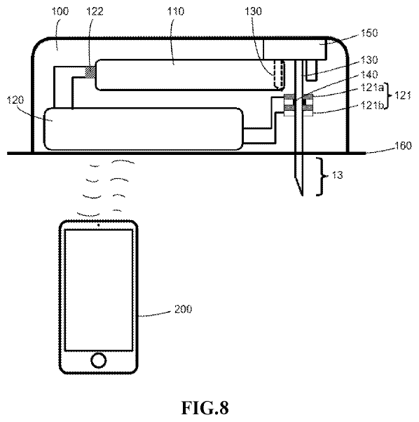

FIG. 7a is a top view of a highly integrated drug infusion device 100 in accordance with another embodiment of the present invention.

In one embodiment of the invention, the integrated drug infusion device 100 comprises two parts. The program unit 120 is disposed in one part, the infusion unit 110 is disposed in another part, and the two parts are electrically connected by the multiple electrical contacts 123. Compared with the plug connector used as connection terminal, the contact area of the electrical contact is much smaller, which provides more flexibility to the structure design, and can effectively reduce the volume of the control structure. At the same time, these smaller electrical contacts can be directly electrically connected to the internal circuit or electrical components, or can be directly soldered on the circuit board, which helps to optimize the design of the internal circuit and effectively reduce the complexity of the circuit, thereby, saving costs and reducing the volume of the infusion device. The type of electrical contact 123 includes rigid metal pins or elastic conductive members, and the type of the elastic conductive member includes conductive spring, conductive silica gel, conductive rubber, or conductive leaf spring.

FIG. 7b is a top view of a highly integrated drug infusion device 100 in accordance with another embodiment of the present invention.

In an embodiment of the invention, the integrated drug infusion device 100 comprises two parts, and the infusion unit 110 comprises two infusion subunits 110a and 110b. The infusion subunits 110a and 110b can be used to reserve different drugs such as insulin, glucagon, antibiotics, nutrient solution, analgesics, morphine, anticoagulants, gene therapy drugs, cardiovascular drugs or chemotherapeutic drugs, etc. Infusion subunits 110a and 110b are electrically coupled to outputs 122a and 122b, respectively, allowing the program unit 120 to effectively control the infusion unit 110. The outlets of infusion subunits 110a and 110b can be connected with the 130a portion and 130b portion of infusion cannula respectively. 130a and 130b are connected with the 130c portion of infusion cannula, respectively. The 130c portion of the infusion cannula is used to penetrate the skin, thereby establishing a path for the two drugs to flow from the infusion unit 110 into the body fluid. That is, the integrated drug infusion device still penetrates the skin only in one position. In the embodiment of the present invention, after the body fluid analyte data signal is transmitted to the program unit 120, program unit 120 can output different infusion signals to different infusion subunits to control whether infusion of drug is required. This method realizes accurate detection and control of body fluid analyte level to stabilize the physiological state of the user.

In other embodiments of the present invention, there may be more infusion subunits according to actual needs, and multiple infusion subunits may be disposed in different parts of the integrated drug infusion device 100. There are no specific restrictions herein.

Referring to FIG. 8, signals are transmitted between the remote device 200 and the integrated drug infusion device 100.

The embodiment of the invention also includes a remote device 200. The remote device 200 includes but is not limited to a handset, a mobile terminal, or the like. The remote device 200 and the program unit 120 transmit wireless signals to each other. Program unit 120 may send body fluid analyte data or drug infusion information (including infusion or no infusion) or warning information to remote device 200. The remote device 200 can receive, record, store, display body fluid information or infusion information or warning information, as well as other functional options. The user can view historical or real-time information at any time from the remote device 200. Through the remote device 200, the user can also manually set the infusion instructions or warning information and transmit the information wirelessly to the program unit 120. Under the premise that the program unit 120 guarantees the communication security and infusion security, the infusion unit is controlled to perform the drug infusion, thereby realizing remote manual control.

In summary, the present invention discloses A highly integrated drug infusion device, one end of the conductor is fixed and electrically connected to the PAD, and the other end is slidably connected to the electrical connection area of the program unit. The sliding connection between the conductor and the electrical connection area replaces the sliding connection between the PAD and the electrical connection area, thus the PAD will not be damaged. And the conductor's thickness is much larger than that of PAD, and its wear resistance is also much larger than that of PAD, so it increases the electrical connection stability and prolongs the service life of the highly integrated drug infusion device.

While the invention has been described in detail with reference to the specific embodiments of the present invention, it should be understood that it will be appreciated by those skilled in the art that the above embodiments may be modified without departing from the scope and spirit of the invention. The scope of the invention is defined by the appended claims.

1. A highly integrated drug infusion device, comprising:
an infusion unit configured to deliver drugs;
a program unit comprises an input end and an output end, and the input end comprises a plurality of electrical connection areas for receiving a signal of analyte data in a body fluid, after the output end is electrically connected to the infusion unit, the program unit controls the drug delivering of the infusion unit;
an infusion cannula provided with electrodes and an electrode contact point; and
a conductor, one end of the conductor is fixed and electrically connected to the electrode contact point, and the other end of the conductor is slidably and electrically connected to the electrical connection areas, when the infusion cannula is installed in a working position, the infusion cannula is connected with the infusion unit, the drugs are able to be injected into the body through the infusion cannula, and the conductor is electrically connected to the electrical connection areas, inputting the signal of analyte data in the body fluid to the program unit.

2. The highly integrated drug infusion device of claim 1, wherein:
the conductor is an elastic conductor.

3. The highly integrated drug infusion device of claim 2, wherein:
the elastic conductor is elastic pins.

4. The highly integrated drug infusion device of claim 2, wherein:
the elastic conductor is an elastic conductive member that comprises spaced conductive area and insulating area.

5. The highly integrated drug infusion device of claim 4, wherein:
the elastic conductive member is a hollow elastic conductive column, which is in interference fit with the infusion cannula.

6. The highly integrated drug infusion device of claim 4, wherein:
the elastic conductive member is a rectangular solid.

7. The highly integrated drug infusion device of claim 6, wherein:
the elastic conductive member at least partially surrounds the infusion cannula.

8. The highly integrated drug infusion device of claim 4, wherein:
the conductive area and the insulating area pass through the elastic conductive member in a transverse direction, respectively.

9. The highly integrated drug infusion device of claim 4, wherein:
the conductive area and the insulating area surround a surface of the elastic conductive member.

10. The highly integrated drug infusion device of claim 4, wherein:
the conductive areas are elastic conductive members distributed at intervals, and the insulating area is the spaced air area of the conductive area.

11. The highly integrated drug infusion device of claim 1, wherein:
the electrical connection area is a metal conductive contact.

12. The highly integrated drug infusion device of claim 4, wherein:
the elastic conductor is one of conductive adhesive strips, conductive foam or conductive bubble.

13. The highly integrated drug infusion device of claim 1, wherein:
the highly integrated drug infusion device consists of multiple parts, the infusion unit and the program unit are arranged in different parts, and the different parts are electrically connected through a plurality of electrical contacts.

14. The highly integrated drug infusion device of claim 1, wherein:
the conductor comprises an elastic pin and an elastic conductor including a spaced conductive area and an insulating area.
